(19) United States
(12) Patent Application Publication
(10) Pub. No.: US 2021/0000730 A1
(43) Pub. Date: Jan. 7, 2021

(54) COMPOSITION COMPRISING OPTICAL CLEARING AGENT FOR IMPROVING OPTICAL CHARACTERISTIC OF SKIN

(71) Applicant: LG HOUSEHOLD & HEALTH CARE LTD., Seoul (KR)

(72) Inventors: Seung-Hyun JUN, Seoul (KR); Seol-Hoon LEE, Seoul (KR); Nae-Gyu KANG, Seoul (KR)

(73) Assignee: LG HOUSEHOLD & HEALTH CARE LTD., Seoul (KR)

(21) Appl. No.: 16/968,826

(22) PCT Filed: Oct. 24, 2018
(86) PCT No.: PCT/KR2018/012667

§ 371 Date: Aug. 10, 2020

(30) Foreign Application Priority Data
Feb. 8, 2018 (KR) 10-2018-0015933
Jun. 11, 2018 (KR) 10-2018-0067069

(51) Int. Cl. A61K 8/73; A61K 8/34; A61K 8/60; A61K 8/41; A61K 8/49; A61K 8/44; A61K 8/19; A61Q 19/00

(52) U.S. Cl. CPC A61K 8/735; A61K 8/345; A61K 8/60; A61Q 19/00; A61K 8/4946; A61K 8/44; A61K 8/19; A61K 8/416

(57) ABSTRACT

The present disclosure relates to a composition for improving optical characteristics of skin, which improves changes of optical characteristics of skin and skin texture, changes of skin tone such as dullness, and the like, caused by progress of keratinization, by applying an optical clearing agent to skin, and thereby, it comprises an optical clearing agent in the composition, thereby having an effect of reducing crystallization of constituents as the optical clearing agent penetrates between keratinocytes, and reducing light scattering as the substance for improving optical characteristics penetrated in the keratinocytes remains in the keratinocytes and binds to water, and through this, improving skin clearing, skin tone and skin texture, and therefore, it may be very usefully used as skin external preparation and cosmetic compositions.

### TECHNICAL FIELD

The present application claims the priority based on Korean Patent Application No. 10-2018-0015933 filed on Feb. 8, 2018 and Korean Patent Application No. 10-2018-0067069 filed on Jun. 11, 2018, and the entire contents disclosed in the description and drawings of the corresponding applications are incorporated in the present application.

The present disclosure relates to a composition for improving optical characteristics of skin, which improves changes of optical characteristics of skin and skin texture, changes of skin tone such as dullness, and the like, caused by progress of keratinization, by applying an optical clearing agent to skin.

### BACKGROUND ART

Corneum is formed by going through a differentiation process of keratinocytes called turn over, whereby keratinocytes existing under the epidermis move to the stratum corneum, and eventually, becomes keratinocytes. As skin cells which were transparent lose moisture during the differentiation process, the cell constituents are crystallized, and as the melanin pigments and the like are close to the skin surface, they lose moisture and are oxidized, and thereby the stratum corneum become opaque and the light is faded, and it looks dull (See FIG. 1).

Therefore, there are products with the concept of inducing exfoliation of corneum to occur in accordance with the turnover period (28 days), products or procedures that exfoliates the outermost corneum by peeling by force, and the like, but there are disadvantages that in the former case, the effect can be obtained only through continuous management, and in the latter case, there is an effect of temporarily clearing skin, but the skin irritation is strong and fundamentally, it is unable to control the transparency of corneum. Many people have been continuously doing skin care by the above method due to the advantage that can be obtained through corneum management (that is, it can improve the skin color and look transparent) despite the above inconvenience. In addition, recently, studies have been conducted to form a thin film with moisture using oil to correct light scattering of skin (See Patent document 1), but there is a disadvantage that it is a temporary method and it is not easy to implement a natural image.

Biological tissue is composed of various materials such as collagen, elastic fibers, cells, extracellular matrix (ECM), and the like. Since these components have different refractive indices, they vary depending on the type of tissue, the degree of dehydration of cells, and the ratio of components, but light scattering of skin tissue is inevitable. Therefore, in optical, optical clearing agents (OCA) such as glycerin, glucose, polyethylene glycol, dimethyl sulfoxide, dimethyl sulfoxide oleic acid, and the like have been used to increase light transmittance, as they are essential for clear and clean analysis of biological tissue by a microscope.

G. Vargas reported the study result that when glycerol is applied or injected by injection to skin, it is helpful to analyze blood vessels in skin (See Non-patent documents 1 and 2). In addition, H. Hiroshi of Japan Riken Research Institute succeeded in treating 4M urea and clearing brain of a mouse (See Non-patent document 3), and K. Meng-Tsen of the same research institute has contributed to bio-optical research by clearing mouse brain using fructose solution of 80% or more (See Non-patent document 4). In general, OCA is known to set the refractive index to be constant due to hydration of ECM, replacement due to absorption of optical clearing agents between tissue cells, and structural changes of collagen, but a clear mechanism has not been identified yet.

Since optics focus on analyzing and diagnosing blood vessels in tissue or treating tattoos, pigmentation, and the like, in skin, using a laser, research to increase the transparency of tissue by delivering the OCA to deep areas of tissue is mainly conducted. For example, a method of inducing clearing of biological tissue by overcoming the skin barrier role of the epidermis (stratum corneum) by various methods such as a method of directly injecting the OCA into the dermis layer, a method of increasing transmittance by damaging or removing the epidermis, delivering the OCA into the dermis layer using ultrasound, and the like, has been studied. However, since side effects of irritation, erythema and severe necrosis of skin have been reported when the OCA is injected to the human body or effective absorption is induced, it is difficult to apply it to stratum corneum clearing by a method used generally used for optical measurement.

To solve these problems, attempts have been made to reduce the concentration of the OCA component and use it on skin. For example, urea has been used by a method of using a content within the allowable limit of the Cosmetics Act. However, there are problems that skin irritation still occurs due to harmfulness of the OCA component itself, or use is limited due to instability in the formulation.

Patent document 1. Korean Patent Publication No. 10-2011-0111968

Non-patent document 1. G. Vargas, Laser Sur. Med. 1999, vol. 24, pp. 133

Non-patent document 2. G. Vargas, Photochem. Photobiol. 2003, vol. 77(5), pp. 541

Non-patent document 3. H. Hiroshi, Nat. Neurosci. 2011, vol. 14(11), pp. 1481

Non-patent document 4. K. Meng-Tsen, Nat. Neurosci. 2013, vol. 16(8), pp. 1154

### DISCLOSURE

### Technical Problem

The present disclosure is to provide a composition which comprises an optical clearing agent in the composition to reduce crystallization of constituents as the optical clearing agent penetrates between keratinocytes, and to reduce light scattering as the substance for improving optical characteristics penetrated in the keratinocytes remains in the keratinocytes and binds to water, and through this, improves skin clearing, skin tone and skin texture.

### Technical Solution

To achieve the aforementioned objects, the present disclosure provides a composition for improving optical characteristics of skin comprising at least one optical clearing agent selected from the group consisting of xylitol, erythritol, sorbitol, trehalose, propanediol, dipropylene glycol, betaine, oligo hyaluronic acid, sodium nitrate (NaNO₃), sodium chloride (NaCl), sodium sulfate (Na₂SO₄), sodium carbonate (Na₂CO₃), potassium nitrate (KNO₃), potassium chloride (KCl), potassium sulfate (K₂SO₄), potassium carbonate (K₂CO₃), ammonium nitrate ((NH₄)(NO₃)), ammonium chloride (NH₄Cl), ammonium sulfate ((NH₄)₂SO₄), ammonium carbonate ((NH₄)₂CO₃), magnesium nitrate (Mg(NO₃)₂.6H₂O), magnesium chloride (MgCl₂), magnesium sulfate (MgSO₄), serine, carnitine and carnosine as an active ingredient.

In one aspect of the present disclosure, the optical clearing agent is characterized by being comprised in an amount of 0.01 to 40% by weight based on the total weight of the composition.

In one aspect of the present disclosure, the oligo hyaluronic acid is characterized by having a molecular weight of 300 to 100,000 Da.

In one aspect of the present disclosure, the optical clearing agent is characterized by comprising a mixture of sorbitol, trehalose and propanediol.

In one aspect of the present disclosure, the mixture of sorbitol, trehalose and propanediol is characterized by comprising sorbitol, trehalose and propanediol at a weight ratio of 1:0.01 to 20:0.01 to 20.

In one aspect of the present disclosure, the optical clearing agent is characterized by having an effect of skin clearing, skin tone improvement or skin texture improvement.

In one aspect of the present disclosure, the composition for improving optical characteristics of skin is characterized by being a cosmetic composition or skin external preparation.

In one aspect of the present disclosure, the cosmetic composition is characterized by being formulated as a formulation selected from the group consisting of water softer, toner water, nutrition water, lotion, essence, eye cream, eye essence, oil serum, serum, cleansing cream, cleansing foam, cleansing water, pack, gel, body wash, body lotion, body oil, body essence, makeup primer, foundation, makeup base, two-way cake, pact, powder, lip stick, lip gloss, lip liner, mascara, eyebrow, eyeshadow and nail enamel.

In one aspect of the present disclosure, the skin external preparation is characterized by being formulated as one selected from the group consisting of cream, gel, ointment, liquid, powder, spray and patch.

### Advantageous Effects

The present disclosure comprises an optical clearing agent in the composition, thereby having an effect of reducing crystallization of constituents as the optical clearing agent penetrates between keratinocytes, and reducing light scattering as the substance for improving optical characteristics penetrated in the keratinocytes remains in the keratinocytes and binds to water, and through this, improving skin clearing, skin tone and skin texture, and therefore, it may be very usefully used as skin external preparation and cosmetic compositions.

### BRIEF DESCRIPITON OF THE DRAWINGS

FIG. 1 is a mimetic diagram showing the interaction of light and each layer of skin.

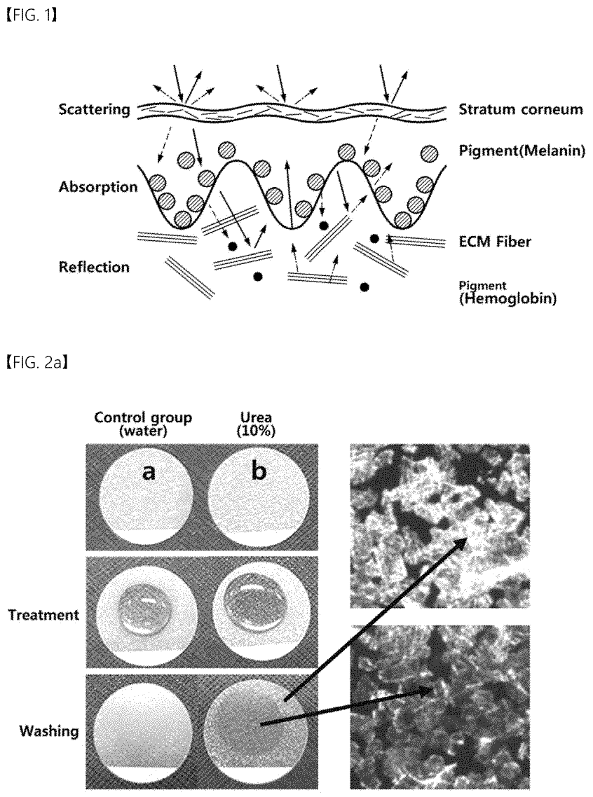

FIG. 2 is (a) corneum tape dried after water treatment and washing and (b) photographs and polarization microscope images (bright field image and dark field image) of the corneum tape dried after treatment of the optical clearing agent and washing, and (c) a graph of the result of light scattering reduction of urea (10%).

FIG. 3 is a graph of relatively comparing the degree of light scattering reduction of the light clearing agents of the present disclosure with the efficiency of light scattering reduction of urea.

FIG. 4 is a graph of comparing the degree of light scattering reduction according to the molecular weight of hyaluronic acid.

FIG. 5 is a graph of comparing the degree of light scattering reduction according to the composition of the single substance and mixture of trehalose, propanediol and sorbitol.

FIG. 6 is the result of measuring the corneum treated by control group 1 and experimental group 11 with a 3D laser microscope.

### MODE FOR INVENTION

The present disclosure for achieving the aforementioned objects is characterized by being a composition for improving optical characteristics of skin comprising at least one optical clearing agent selected from the group consisting of xylitol, erythritol, sorbitol, trehalose, propanediol, dipropylene glycol, betaine, oligo hyaluronic acid, sodium nitrate (NaNO₃), sodium chloride (NaCl), sodium sulfate (Na₂SO₄), sodium carbonate (Na₂CO₃), potassium nitrate (KNO₃), potassium chloride (KCl), potassium sulfate (K₂SO₄), potassium carbonate (K₂CO₃), ammonium nitrate ((NH₄)(NO₃)), ammonium chloride (NH₄Cl), ammonium sulfate ((NH₄)₂SO₄), ammonium carbonate ((NH₄)₂CO₃), magnesium nitrate (Mg(NO₃)₂.6H₂O), magnesium chloride (MgCl₂), magnesium sulfate (MgSO₄), serine, carnitine and carnosine as an active ingredient. Hereinafter, the present disclosure will be described in detail with reference to drawings.

The term used herein, ‘skin light scattering’ means that when external light penetrates into skin, light is not scattered and dispersed in the stratum corneum in skin and penetrates into the epidermis and dermis, and then reflects out of the skin. The meaning of increasing skin light scattering means that the ratio that light which penetrates into skin is reflected again is high.

When a composition such as a cosmetic or external preparation is applied to skin, various properties such as skin moisture content, skin surface roughness and skin condition are changed. Each parameter is an important factor that changes the behavior (refractive index, reflection, path difference) of light on skin.

The present disclosure provides a composition for improving optical characteristics of skin comprising at least one optical clearing agent selected from the group consisting of xylitol, erythritol, sorbitol, trehalose, propanediol, dipropylene glycol, betaine, oligo hyaluronic acid, sodium nitrate, sodium chloride, sodium sulfate, sodium carbonate, potassium nitrate, potassium chloride, potassium sulfate, potassium carbonate, ammonium nitrate, ammonium chloride, ammonium sulfate, ammonium carbonate, magnesium nitrate, magnesium chloride, magnesium sulfate, serine, carnitine and carnosine as an active ingredient.

The optical clearing agent comprised in the composition of the present disclosure can be evenly delivered to the stratum corneum that is the outermost layer of skin to reduce crystallization of constituents, and reduce light scattering as the substance for improving optical characteristics which penetrates into keratinocytes remains in the keratinocytes and binds to moisture, and through this, make the skin look more transparent and improve skin tone and skin texture. The ‘skin texture improvement’ means that the stratum corneum is dried and is not floated, and thereby skin softness is increased, improved or enhanced, or roughness is reduced. The ‘skin tone improvement’ means the degree of dynamics, or darkness or lightness of own skin color. Therefore, it means that the substance for improving optical characteristics improves dullness of skin corneum and it is improved as clean skin as it is.

In the composition of the present disclosure, the optical clearing agent may be comprised in an amount of 0.01 to 40% by weight, specifically, 0.1 to 30% by weight, more specifically, 1 to 20% by weight, based on the total weight of the composition. When the optical clearing agent is comprised less than 0.01% by weight, the effect of improving optical characteristics of skin may be insignificant, and when it is comprised over 40% by weight, skin may be hard and deformed, and the skin may be overly transparent, which may harm natural skin shine.

In the composition of the present disclosure, the oligo hyaluronic acid may have a molecular weight of 300 to 100,000 Da, specifically, 300 to 50,000 Da, more specifically, 500 to 10,000 Da. When the molecular weight of the oligo hyaluronic acid is outside the above range, the efficacy of light scattering reduction and effect of improving optical characteristics of skin may be lowered.

In the composition of the present disclosure, the optical clearing agent may comprise a mixture of sorbitol, trehalose and propanediol.

In the composition of the present disclosure, the mixture of sorbitol, trehalose and propanediol may comprise sorbitol, trehalose and propanediol at a weight ratio of 1:0.01 to 20:0.01 to 20, specifically, a weight ratio of 1:0.1 to 15:0.1 to 15, more specifically, a weight ratio of 1:0.5 to 10:0.5 to 10.

In the composition of the present disclosure, the optical clearing agent has an effect of skin clearing, skin tone improvement or skin texture improvement.

In the composition of the present disclosure, the composition for improving optical characteristics of skin may be a cosmetic composition or skin external preparation.

The cosmetic composition of the present disclosure may be used as a component of a general cosmetic or functional cosmetic, with common adjuvants, for example, adjuvants commonly used in the cosmetic or skin science field such as not only fat substances, organic solvents, solvents, concentrates, gelling agents, softeners, anti-oxidants, suspending agents, stabilizers, foaming agents, air fresheners, surfactants, water, ionic or non-ionic emulsifiers, fillers, metal ion sequestering agents, chelating agents, preservatives, vitamins, blocking agents, wetting agents, essential oils, dyes, pigments, hydrophilic or lipophilic activators, lipid vesicles, but also any other component commonly used for cosmetics.

The cosmetic composition of the present disclosure may be formulated as a common cosmetic formulation, and as one example, it may be formulated as a formulation selected from the group consisting of water softer, toner water, nutrition water, lotion, essence, eye cream, eye essence, oil serum, serum, cleansing cream, cleansing foam, cleansing water, pack, gel, body wash, body lotion, body oil, body essence, makeup primer, foundation, makeup base, two-way cake, pact, powder, lip stick, lip gloss, lip liner, mascara, eyebrow, eyeshadow and nail enamel.

The skin external preparation of the present disclosure may contain common adjuvants, for example, adjuvants commonly used in the cosmetic or skin science field such as any other component commonly used for skin external preparations such as fat substances, organic solvents, solvents, concentrates, gelling agents, softeners, anti-oxidants, suspending agents, stabilizers, foaming agents, air fresheners, surfactants, water, ionic or non-ionic emulsifiers, fillers, metal ion sequestering agents, chelating agents, preservatives, vitamins, blocking agents, wetting agents, essential oils, dyes, pigments, hydrophilic or lipophilic activators, lipid vesicles. In addition, the above components may be introduced in a commonly used amount in the skin science field.

The skin external preparation of the present disclosure may be formulated as a common skin external application formulation, and as one example, it may be formulated as a formulation selected from the group consisting of cream, gel, ointment, liquid, powder, spray and patch.

Hereinafter, the present disclosure will be described in more detail by examples. However, they are intended to illustrate the present disclosure in more detail, but the scope of the present disclosure is not limited thereto.

### EXAMPLE 1

The corneum of subjects was collected using corneum tape (D-Squame sampling disk, 22 mm diameter, CuDerm™), and then 100 ul of water (control group) and urea were applied respectively, and in 3˜4 hours, it was dried after washing and the effect of light scattering was confirmed.

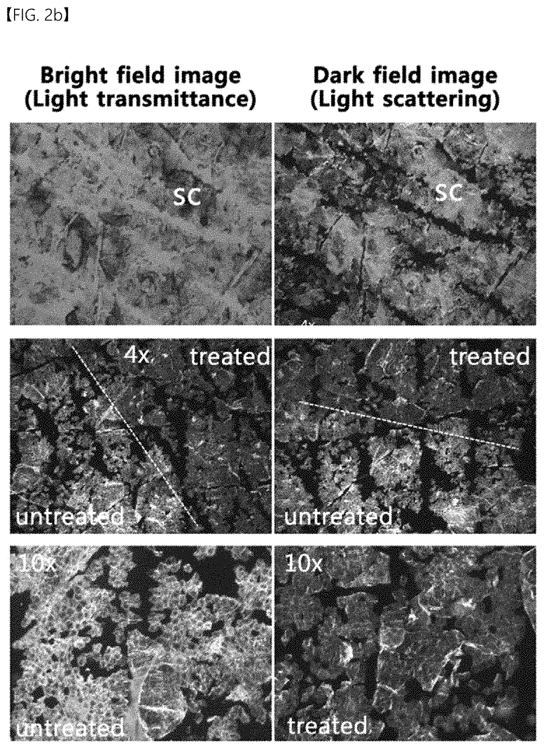

The experimental result was illustrated in FIGS. 2a to 2c. As the result of the experiment, it could be confirmed that when the control group (water) was applied and washed and then dried, it did not remain in the corneum and the reduction of scattering was not shown, but on the other hand, as the urea penetrated into the keratinocytes and held water molecules even though it was treated and washed and then dried, the scattering was reduced and it appeared transparent (the right bottom picture of FIG. 2a).

FIG. 2b shows the bright field image and dark field image of the polarization microscope (Leica DM 1000) when treating urea (10%). The bright field image is a basic observation method of simply magnifying a sample without a unique optical unit and it is suitable for confirming the structure of the sample, but it is not appropriate for confirming the scattering light. The dark field image is an observation method of inducing the direct illumination light reaching the sample out, and collecting only the scattering light from the sample with objective lens, and the light from the upper light source of the microscope is scattered from the keratinocytes and appears white, and as the scattering decreases, it appears black. As a result, it was confirmed that the corneum scattering was reduced and appeared black when urea was treated.

FIG. 2c is the result of obtaining 5 sites of the applied site and unapplied site of the sample from the corneum tape as images through the dark field mode of the polarization microscope, and then comparing the light scattering reduction efficiency by digitizing using an image analysis program (Python). The numerical value of the analyzed image is expressed in the range of 30-200, and the more the white image is, the higher the value is. The average value of the image numerical value of the unapplied site and the average value of the image numerical value of the applied site were obtained, and the reduction ratio of the image numerical value of the applied site compared to the unapplied site was calculated as a percentage. As the result of the experiment, it was confirmed that the reduction of light scattering was 17% compared to the control group (water), when 10% of urea was treated.

### EXAMPLE 2

For the composition comprising an optical clearing agent, that is propanediol, erythritol, xylitol, sorbitol, trehalose, dipropylene glycol, betaine, carnitine, carnosine and serine in an amount of 10% by weight of the present disclosure, using the same method as Example 1 described above, the effect of reduction of light scattering was confirmed.

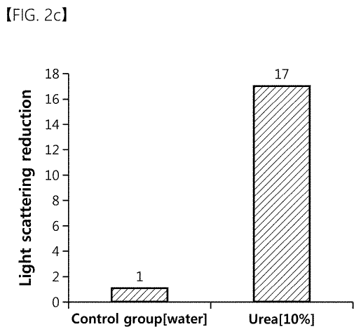

On the other hand, to compare the effect of the present disclosure by relative values at the same concentration, the solution of urea, glycerol, sucrose, sodium lactate, polyethylene glycol 400 and hexanediol of the same concentration (10% by weight) was prepared and compared and evaluated (FIG. 3a). Oligo hyaluronic acid (500˜10,000 Da) and sodium chloride were prepared as a 1% by weight solution and compared and evaluated with urea at the same concentration (1% by weight) (FIG. 3b).

The experimental result was shown in FIG. 3a by representing the efficiency of reduction of light scattering obtained by analyzing the image obtained by the polarization microscope as a relative value with the efficiency of reduction of light scattering of urea (10% by weight) as 100%, and it was shown in FIG. 3a by representing it as a relative value with the efficiency of reduction of light scattering of urea (1% by weight) as 100%.

As the result of the experiment, it could be confirmed that all the optical clearing agents of the present disclosure showed the significant efficiency of reduction of light scattering, respectively, and among them, it was confirmed that the oligo hyaluronic acid, trehalose, propanediol, sorbitol, dipropylene glycol and sodium chloride particularly showed the efficiency of reduction of scattering similar to or higher than glycerol, sucrose, sodium lactate, polyethylene glycol 400 and hexanediol as well as urea.

### EXAMPLE 3

To confirm the effect of the reduction of light scattering of the oligo hyaluronic acid, the optical clearing agent of the present disclosure, the comparative experiment was conducted with hyaluronic acid having a different molecular weight.

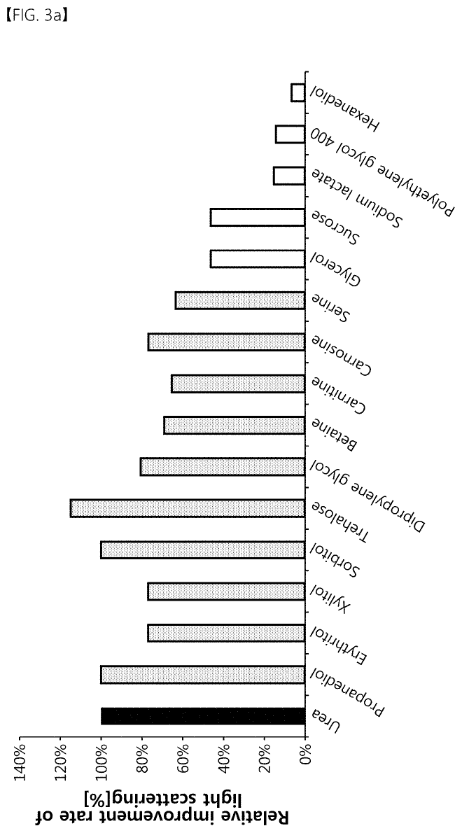

Specifically, using the same method as Example 1 described above, the substances of the following Table 1 were treated, and the result values were shown in FIG. 4 by representing it as relative values with the efficiency of reduction of light scattering of urea as 100%

TABLE 1

| Classification | Substance | Concentration |
|---|---|---|
| Experimental | Hyaluronic acid having a molecular | 1% by |
| group 1 | weight of 300,000 Da | weight |
| | or more (500,000 Da) | |
| Experimental | Hyaluronic acid having a molecular | 1% by |
| group 2 | weight of 100,000~300,000 | weight |
| | Da or more (100,000 Da) | |
| Experimental | Hyaluronic acid having a molecular | 1% by |
| group 3 | weight of 10,000 Da or | weight |
| | less (500~10000 Da) | |

As the result of the experiment, it was confirmed that the relative reduction ratio of light scattering of experimental groups 1 and 2 stayed at around 50%, while the reduction ratio of light scattering of experimental group 3 that was hyaluronic acid having a molecular weight of 10,000 Da or less was shown very high as 18% (FIG. 4). This result may be considered as hyaluronic acid having a small molecular weight penetrates into keratin in the corneum and holds water.

### EXAMPLE 4

The effect of reduction of light scattering depending on the mixing ratio of trehalose, propanediol and sorbitol that were optical clearing agents of the present disclosure was experimented.

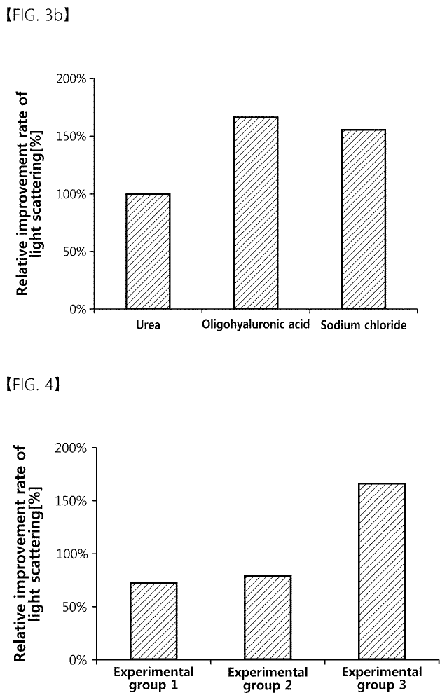

As the specific experimental method, the same method as experimental example 1 was used, and the composition of the following Table 2 was treated (content was % by weight), and the result values were shown in FIG. 5 by representing them as relative values with the efficiency of reduction of light scattering of urea as 100%.

TABLE 2

| | Experimental | Experimental | Experimental | Experimental | Experimental | Experimental | Experimental |
|---|---|---|---|---|---|---|---|
| | group 4 | group 5 | group 6 | group 7 | group 8 | group 9 | group 10 |
| Sorbitol | 0 | 0 | 10 | 7 | 1 | 2 | 3.3 |
| Propanediol | 0 | 10 | 0 | 2 | 7 | 1 | 3.3 |
| Trehalose | 10 | 0 | 0 | 1 | 2 | 7 | 3.3 |
| Purified | up to 100 | up to 100 | up to 100 | up to 100 | up to 100 | up to 100 | up to 100 |
| water | | | | | | | |

As the result of the experiment, it was confirmed that the efficiency of reduction of scattering was significantly increased in experimental groups 4 to 7 in which each substance was combined all, compared to experimental groups 1 to 3 in which each single substance was treated, and also the improvement rate of light scattering represented by the relative value compared to urea was significantly high, and therefore there was a significant synergistic effect (FIG. 5).

### EXAMPLE 5

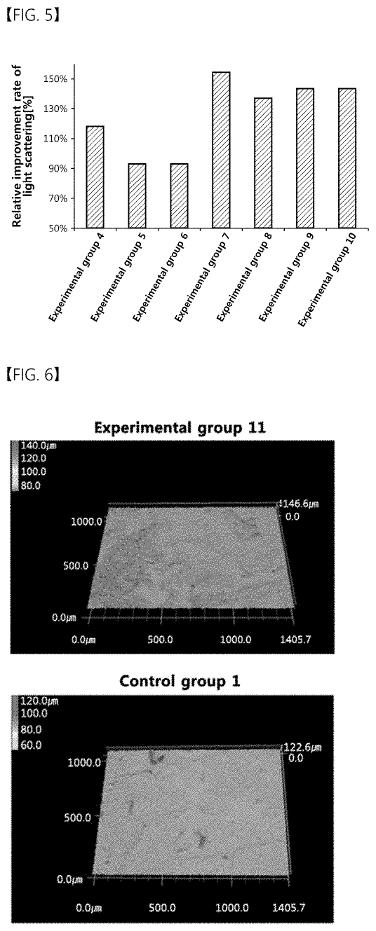

To confirm the skin texture improvement of the present disclosure, the corneum of subjects was collected using corneum tape, and then 200 ul of water (control group 1) and experimental group 11 (See the following table) were applied and in 4 hours, it was washed with water and dried. For the skin texture improvement, the surface thickness and surface roughness were measured using a 3-dimensional laser microscope (Keyence VK-X200). The experimental result was shown in the following table and FIG. 6.

TABLE 3

| | Experimental group 11 | Control group 1 | Note |
|---|---|---|---|
| Composition | Propanediol 7%, | Water | |
| | Trehalose 2%, | | |
| | Sorbitol 1% | | |
| | Purified water | | |
| | up to 100 | | |
| Corneum | 16.3 | 14.2 | About 15% |
| thickness | | | increase |
| (μm) | | | |
| Roughness | 3.2 | 3.8 | About 14% |
| (μm) | | | decrease |

Specifically, it could be confirmed that the corneum thickness was increased by 15% and the roughness was decreased by 14% in the sample treated with experimental group 11 compared to the control group. This may be considered that the optical clearing agent of the present disclosure penetrates into keratinocytes and holds moisture after drying, and therefore the thickness of the corneum is increased, and thereby the roughness is decreased.

### EXAMPLE 6

To confirm the skin transparency improvement of the present disclosure, control group 2 and experimental groups 12 and 13 (See the following table) were applied to 10 subjects, and in 3 hours, after washing with a body wash, the skin transparency, which was different from before application, was evaluated using a skin transparency meter (TLS850).

TABLE 4

| | | | | | Rate of |
|---|---|---|---|---|---|
| | | | | | change of |
| | | | | | K value |
| | Glycerin | Propanediol | Trehalose | Sorbitol | (%) |
| Control | 10 | 0 | 0 | 0 | 0.17 |
| group 2 | | | | | |
| Experimental | 0 | 2 | 7 | 1 | 1.76 |
| group 12 | | | | | |
| Experimental | 0 | 7 | 2 | 1 | 2.03 |
| group 13 | | | | | |

The experimental result was shown in Table 4, and it was confirmed that the skin transparency was greatly improved when experimental groups 12 and 13 were treated, compared to control group 2.

In the above, the applicant has described preferable examples of the present disclosure, but these examples are only one example that implements the technical spirit of the present disclosure, and any modification or alteration should be interpreted as belonging to the scope of the present disclosure as long as it implements the technical spirit of the present disclosure.

### INDUSTRIAL APPLICABILITY

The present disclosure comprises an optical clearing agent in the composition, thereby having an effect of reducing crystallization of constituents as the optical clearing agent penetrates between keratinocytes, and reducing light scattering as the substance for improving optical characteristics penetrated in the keratinocytes remains in the keratinocytes and binds to water, and through this, improving skin clearing, skin tone and skin texture, and therefore, it may be very usefully used as skin external preparation and cosmetic compositions.

1. A method for improving optical characteristics of skin comprising administering a composition comprising at least one optical clearing agent selected from the group consisting of xylitol, erythritol, sorbitol, trehalose, propanediol, dipropylene glycol, betaine, oligo hyaluronic acid, sodium nitrate (NaNO₃), sodium chloride (NaCl), sodium sulfate (Na₂SO₄), sodium carbonate (Na₂CO₃), potassium nitrate (KNO₃), potassium chloride (KCl), potassium sulfate (K₂SO₄), potassium carbonate (K₂CO₃), ammonium nitrate ((NH₄)(NO₃)), ammonium chloride (NH₄Cl), ammonium sulfate ((NH₄)₂SO₄), ammonium carbonate ((NH₄)₂CO₃), magnesium nitrate (Mg(NO₃)₂.6H₂O), magnesium chloride (MgCl₂), magnesium sulfate (MgSO₄), serine, carnitine and carnosine as an active ingredient to a subject in need thereof.

2. The method according to claim 1, wherein the optical clearing agent is comprised in an amount of 0.01 to 40% by weight based on the total weight of the composition.

3. The method according to claim 1, wherein the oligo hyaluronic acid has a molecular weight of 300 to 100,000 Da.

4. The method according to claim 1, wherein the optical clearing agent comprises a mixture of sorbitol, trehalose and propanediol.

5. The method according to claim 4, wherein the mixture of sorbitol, trehalose and propanediol comprises sorbitol, trehalose and propanediol at a weight ratio of 1:0.01 to 20:0.01 to 20.

6. The method according to claim 1, wherein the optical clearing agent has an effect of skin clearing, skin tone improvement or skin texture improvement.

7. The method according to claim 1, wherein the composition is a cosmetic composition or skin external preparation.

8. The method according to claim 7, wherein the cosmetic composition is formulated as one selected from the group consisting of water softer, toner water, nutrition water, lotion, essence, eye cream, eye essence, oil serum, serum, cleansing cream, cleansing foam, cleansing water, pack, gel, body wash, body lotion, body oil, body essence, makeup primer, foundation, makeup base, two-way cake, pact, powder, lip stick, lip gloss, lip liner, mascara, eyebrow, eyeshadow and nail enamel.

9. The method according to claim 7, wherein the skin external preparation is formulated as one selected from the group consisting of cream, gel, ointment, liquid, powder, spray and patch.
